(19) United States
(12) Patent Application Publication
(10) Pub. No.: US 2020/0163662 A1
(43) Pub. Date: May 28, 2020

(54) Retractor

(71) Applicants: TOKUSEN KOGYO CO., LTD., Hyogo (JP); OSAKA UNIVERSITY, Osaka (JP)

(72) Inventors: Kiyokazu Nakajima, Osaka (JP); Suekatsu Fujimoto, Hyogo (JP); Keisuke Hirano, Hyogo (JP)

(21) Appl. No.: 16/630,303

(22) PCT Filed: Jul. 5, 2018
(86) PCT No.: PCT/JP2018/025603

§ 371 Date: Jan. 10, 2020

(30) Foreign Application Priority Data
Jul. 11, 2017 (JP) 2017-135192

(51) Int. Cl. A61B 17/02

(52) U.S. Cl. CPC A61B 17/0218; A61B 2017/00867

(57) ABSTRACT

A developing body includes: an excluding portion including a movable wire and fixing wires arranged around the movable wire, a distal end of the movable wire and distal ends of the fixing wires being connected to each other; an insertion tube allowing the movable wire to pass therethrough; and a restriction member that is connected to a proximal end of the movable wire. The restriction member restricts the movement of the movable wire when the restriction member abuts against the first grip portion. When the first grip portion and the second grip portion are moved relative to each other so as to be close to each other in this state, the excluding portion is developed by bringing the distal end of the insertion tube and the distal end of the movable wire to be close to each other to deflect the fixing wire.

### TECHNICAL FIELD

The present invention relates to retractors, and more particularly, to a retractor deployable in a three-dimensional direction which is used to exclude an inside wall of a hollow organ or an organ in a body cavity.

### BACKGROUND

It is known that the organ which is an obstacle to surgery can be manually spread in a conventional laparotomy, but in endoscopic surgery, the organ cannot be spread easily, and a field of view optimum for the surgery is difficult to ensure. In an example method, gas is injected into the body cavity to ensure the field of view and operation space. This method, however, requires general anesthesia, and accordingly, would be highly invasive as the endoscopic surgery.

To alleviate the problem associated with the field of view and facilitate endoscopic surgery, an instrument referred to as a retractor has been developed that excludes or tugs a to-be-treated organ or an organ which is an obstacle to the field of view during a treatment. It is desired that, as a basic function, the retractor can allow the instrument to be inserted therethrough via a small opening passage that serves as an insertion passage, such as a trocar (over-tube) or a small incision, in the insertion of the retractor into the body. Thus, the retractor needs to have a small diameter (e.g., desirably 10 mm or less in the case of trocar and 20 mm or less in the case of small incision) and be in the form of a rod at least in its insertion, whereas after the insertion into the body cavity, the retractor is required to be deformable into a shape in which an excluding portion has a large area enough to widely and effectively exclude the target.

Variously devised retractors are known that respond to the above conflicting demands. For example, Japanese Patent Laying-Open No. 06-154152 proposes a medical exclusion hook (retractor) including an opening and closing portion movable to form a scoop, a rod, and a grip. When the grip is grasped, the movement of the grip slides an inner rod in the rod and opens the opening and closing portion, thereby forming a fan-shaped scoop.

WO 2015/079719 proposes a retractor including a rigid perforation tube, a developing body configured to be accommodated and extended with respect to the perforation tube, and a grip connected to respective proximal ends of the perforation tube and the developing body. In the retractor of WO 2015/079719, the developing body includes an excluding section configured by a movable wire and a plurality of fixing wires arranged at a periphery of the movable wire, and an insertion tube which is extended from the excluding section, the insertion tube allowing the movable wire to pass therethrough. The grip includes a first grip portion, a second grip portion, and a third grip portion from a distal side. A proximal end of the perforation tube is connected to the first grip portion, a proximal end of the insertion tube is connected to the second grip portion, and a proximal side of the movable wire is connected to the third grip portion. In the retractor of WO 2015/079719, the second grip portion and the third grip portion are pushed in toward the distal end side, that is, the first grip portion side with respect to the first grip portion. Thus, the insertion tube and the movable wire connected to the second grip portion and the third grip portion, respectively, are pushed out toward the distal side, and the developing body is extended from the perforation tube. With the developing body extended from the perforation tube, the fixing wires are curved toward the outer side by pulling out only the third grip portion toward the proximal side (hand side) with respect to the first grip portion and the second grip portion, or pushing out only the second grip portion with respect to the first grip portion and the third grip portion, so that the excluding section is developed.

Although the retractor of WO 2015/079719 enables the exclusion of an organ interfering with an operative field by operating the first to third grip portions to develop the excluding section as described above, the first to third grip portions each need to be operated, leading to a desire for further improvements in operability.

### SUMMARY OF THE DISCLOSURE

The present invention has been made to solve the above problem, and has an object to provide a retractor easily handled in endoscopic surgery.

To solve the above problem, a retractor of the present invention includes:

an outer tube having a tube shape;

a developing body inserted into the outer tube;

a first grip portion connected to a proximal end of the outer tube; and

a second grip portion that is connected to the developing body and moves relative to the first grip portion to move the developing body and the first grip portion relative to each other, wherein

the developing body includes
an excluding portion including a movable wire and a plurality of fixing wires arranged around the movable wire, a distal end of the movable wire and distal ends of the fixing wires being connected to each other,
an insertion tube having a distal end connected to proximal ends of the fixing wires and a proximal end connected to the second grip portion, the insertion tube allowing the movable wire to pass therethrough, and
a restriction member that is connected to a proximal end of the movable wire and restricts a movement of the movable wire,

the restriction member included in the developing body restricts the movement of the movable wire when the developing body and the first grip portion move relative to each other through the relative movement of the first grip portion and the second grip portion to cause the restriction member to abut against the first grip portion, and

when the first grip portion and the second grip portion are moved relative to each other so as to be close to each other with the movement of the movable wire restricted, the excluding portion is developed by bringing the distal end of the insertion tube connected with the second grip portion and the distal end of the movable wire, the movement of which is restricted, close to each other to deflect the fixing wire.

With this configuration, in the retractor of the present invention, when the second grip portion is moved to the distal side, the movement of the movable wire is restricted by the restriction member, and the excluding portion can be developed by further moving the second grip portion to the first grip portion side with the movement of the movable wire restricted. In other words, there is no need to provide a grip portion for operating the movable wire separately from the first grip portion and the second grip portion, and the excluding portion can be developed by two grip portions, the first grip portion and the second grip portion, thereby facilitating handling in exclusion.

When the first grip portion and the second grip portion are moved relative to each other so as to be close to each other with the developing body accommodated in the outer tube, the developing body connected with the second grip portion and the outer tube connected with the first grip portion may be moved relative to each other to cause the developing body to extend from a tip of the outer tube.

With this configuration, the retractor of the present invention can perform an operation of extending the developing body from the outer tube with two portions, the first grip portion and the second grip portion.

The first grip portion and the second grip portion may be slidably fitted with each other, and the first grip portion and the second grip portion may be moved relative to each other through sliding of the first grip portion and the second grip portion.

With this configuration, the retractor of the present invention can easily move the first grip portion and the second grip portion relative to each other.

The retractor may include a lock mechanism that locks the second grip portion at a position at which the second grip portion is moved to a distal side and the excluding portion is developed.

With this configuration, the retractor of the present invention can maintain the state in which the excluding portion is developed even when the operator does not maintain the position of the second grip portion, thereby performing exclusion more easily.

In the retractor, the outer tube may be a perforation tube having a distal end projecting to a distal side at an acute angle.

With this configuration, the retractor of the present invention can be inserted into the body through direct perforation.

The present invention can provide a retractor easily handled in endoscopic surgery.

### BRIEF DESCRIPTION OF DRAWINGS

FIG. 1 shows an example retractor, where FIG. 1(A) shows a state in which a developing body is accommodated in a perforation tube, FIG. 1(B) shows a state in which the developing body is extended from the tip of the perforation tube, and FIG. 1(C) shows a state in which an excluding portion of the developing body is opened.

FIG. 2 schematically shows an example developing body of the retractor.

FIG. 3 schematically shows example cross-sections of a movable wire and fixing wires in a direction orthogonal to a long axis of the developing body of the retractor.

FIG. 4 schematically shows example cross-sections of the fixing wire.

FIG. 5 is a schematic view of a retractor according to an embodiment as seen from the distal end side of the retractor when the developing body is extended from the perforation tube and developed.

FIG. 6 schematically shows cross-sections of a retractor, where FIG. 6(A) shows a state in which the developing body is accommodated in the perforation tube, FIG. 6(B) shows a state in which the developing body is extended from the tip of the perforation tube, and FIG. 6(C) shows a state in which the excluding portion of the developing body is opened.

FIG. 7 is an exploded perspective view of the retractor.

FIG. 8 shows proximal ends of an insertion tube and a movable wire and therearound, where FIG. 8(A) shows a state in which a restriction member does not restrict a movement of the movable wire, FIG. 8(B) shows a state in which the restriction member restricts a movement of the movable wire, and FIG. 8(C) shows a state in which the second grip portion is moved further to the distal side from the state shown in FIG. 8(B).

FIG. 9 shows an example retractor according to a second embodiment, where FIG. 9(A) shows a state in which a developing body 220 is accommodated in a perforation tube 110, FIG. 9(B) shows a state in which developing body 220 is extended from the tip of perforation tube 110, and FIG. 9(C) shows a state in which an excluding portion 222 of developing body 220 is opened.

FIG. 10 shows example cross-sections of an excluding portion (cross-sections of a movable wire and fixing wires) in a direction orthogonal to a long axis of a developing body of the retractor according to the second embodiment.

FIG. 11 shows a state in which the excluding portion is opened in detail.

FIG. 12 schematically shows an example distal end tip of the retractor according to the second embodiment when the developing body is extended from the perforation tube and developed in the retractor.

### DETAILED DESCRIPTION OF EMBODIMENTS

Preferred embodiments of the present invention will now be described with reference to the drawings. Note that the embodiments described below are given as examples for carrying out the present invention, and the present invention is not limited to the embodiments described below.

### First Embodiment

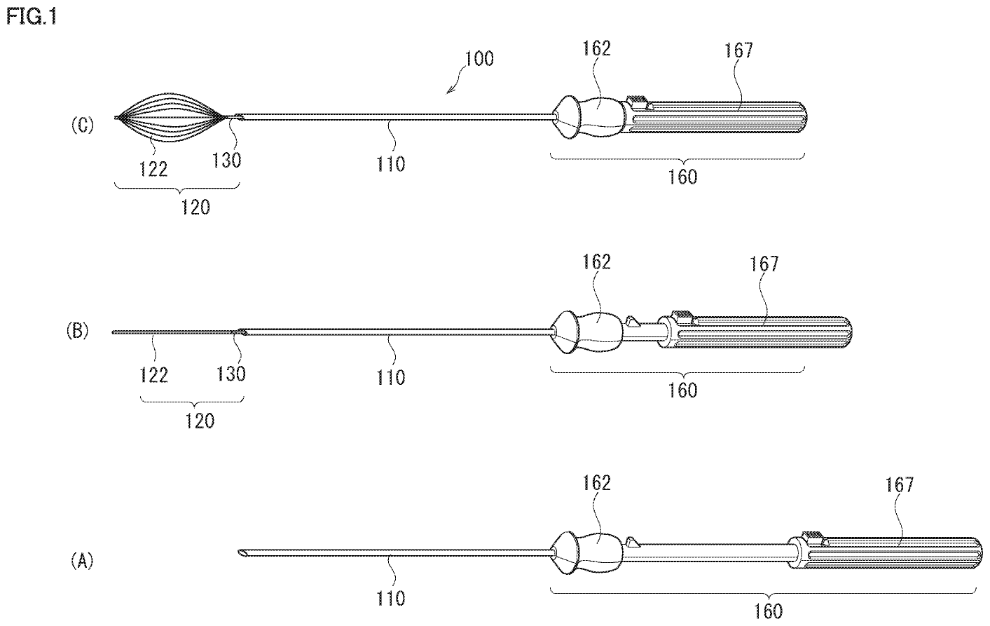

FIG. 1 shows an example retractor 100 according to the present embodiment, where FIG. 1(A) shows a state in which a developing body 120 is accommodated in a perforation tube 110, FIG. 1(B) shows a state in which developing body 120 is extended from the tip of perforation tube 110, and FIG. 1(C) shows a state in which an excluding portion 122 of developing body 120 is opened.

As shown in FIG. 1, retractor 100 includes rigid or hard perforation tube 110, developing body 120 inserted into perforation tube 110 to be accommodated therein and extended therefrom, and a grip 160 connected to the proximal end of perforation tube 110 and developing body 120.

The term “retractor” used herein refers to a medical instrument for excluding, spreading, tugging, or elevating a target (e.g., the organ in the body or an obstacle to a field of view), and may include a trocar and an over-tube. The excluding, spreading, tugging, and elevating operations may be collectively referred to as “retraction” or “retract”. Mere “exclusion” herein may include the excluding operation, as well as the spreading, tugging, and elevating operations (i.e., refers to retraction).

The term “distal” herein refers to a position far from the operator of the retractor, and the term “proximal” refers to a position closer to the operator of the retractor than the “distal” is to the operator. Thus, the term “distal end” refers to an end farthest from the operator and the term “proximal end” refers to an end closest (i.e., proximal) to the operator in the operation of the retractor of the present invention.

Referring to FIG. 1(A) again, perforation tube 110 is formed of a straight tube with a distal end side cut into a sharp-pointed shape. Although the angle of the distal end tip of perforation tube 110 is not particularly limited, the distal end tip is processed to have such an angle (e.g., 20° to 50°) that allows easy perforation into the body. The outside diameter of perforation tube 110 is preferably 1.6 mm to 3.6 mm, more preferably 2.2 mm to 3 mm, and is 2.6 mm in the present embodiment. Further, with respect to the outside diameter, the inside diameter of perforation tube 110 can be selected from the range of preferably 1.3 mm to 3.3 mm, more preferably 1.9 mm to 2.7 mm, and is 2.3 mm in the present embodiment. The shape of the distal end tip of perforation tube 110 is not necessarily limited to the above shape in the present invention and may be any shape that can be adopted in the over-tube or the trocar in the medical field. Perforation tube 110 is not limited to have a precisely straight shape, and it may be designed to have a linear shape as appropriate. Perforation tube 110 of the present embodiment is one embodiment of the outer tube.

Such perforation tube 110 is preferably made of a highly rigid material, for example, a metal such as stainless steel, tantalum, cobalt alloy, and nitinol (nickel-titanium alloy). Examples of the stainless steel include SUS304, SUS316, and SUS316L. Further, in order to prevent sparks generated between perforation tube 110 and another instrument during the surgery, a coating material having electrical insulating properties may be applied to the surface of perforation tube 110. The coating material may be a material normally used for coating of a medical instrument. Examples of the coating material include an expanded polytetrafluoroethylene (ePTFE) film, a silicone film, a polyurethane film, and a polyethylene terephthalate (Dacron, registered trademark) film. Although the thickness of the coating layer formed of the coating material is not particularly limited, it is, for example, 19 μm to 31 μm, preferably 23 μm to 27 μm. In the present embodiment, a coating of 25 μm is applied. Retractor 100 of the present invention can reliably perforate a desired position (e.g., abdominal cavity) with the use of perforation tube 110 formed of such a highly rigid material, without fear of deflection, bend, breakage, or the like of the perforation tube. Further, owing to the rigidity of perforation tube 110, a sufficient strength can be maintained also in the cocoon-shaped development and retraction at the excluding portion, which will be described below. In the present embodiment, the term “cocoon shape” refers to a shape formed as a result of a plurality of fixing wires, which will be described below, curving in the excluding portion of the developing body, and includes, for example, the shapes of a cocoon, an oval sphere, and a rugby ball. The term “cocoon shape” may be referred to as a “perfect cocoon shape” for differentiation from a “partial cocoon shape” described below.

Grip 160 is divided into two portions, namely, a first grip portion 162 and a second grip portion 167 sequentially from the distal end side. The type of the material for grip 160 is not particularly limited. Grip 160 is made of, for example, a resin such as ABS resin, polycarbonate resin, and acryl resin, a metal such as stainless steel and aluminum, and a combination thereof.

In retractor 100 of the present embodiment, for example, developing body 120 is accommodated in perforation tube 110 immediately before use, as shown in FIG. 1(A).

Retractor 100 of the present invention can push and pull second grip portion 167 of grip 160 into and from first grip portion 162 to take in and out developing body 120 accommodated in perforation tube 110. For example, as second grip portion 167 is pushed toward the distal end from the state of FIG. 1(A) to approach first grip portion 162, excluding portion 122 that is the distal portion of developing body 120 extends from the tip of perforation tube 110, as shown in FIG. 1(B).

Developing body 120 is designed to have such a size that includes an outside diameter of preferably 1 mm to 3 mm, more preferably 1.4 mm to 2.2 mm and allows developing body 120 to slide freely in perforation tube 110 when being accommodated in perforation tube 110 (in the state in which development described below is not performed). Developing body 120 is 1.8 mm in the present embodiment. The overall length of the retractor of the present invention is not necessarily limited. For example, the distance from the distal end to the proximal end of perforation tube 110 (i.e., the distance from the distal end of perforation tube 110 to the distal end of first grip portion 162) is preferably 100 mm to 300 mm.

Retractor 100 of the present invention can further push second grip portion 167 in grip 160 toward first grip portion 162 from the state of FIG. 1(B) to develop excluding portion 122 of developing body 120, as shown in FIG. 1(C). Developing body 120 includes excluding portion 122 and an insertion tube 130 extended from excluding portion 122. Excluding portion 122 is composed of a movable wire 124, a plurality of fixing wires 126 arranged around movable wire 124, and a cap 132 connecting movable wire 124 and the fixing wires to each other. Insertion tube 130 has a tubular shape, and movable wire 124 is slidably passed therethrough. Although the number of fixing wires 126 arranged around movable wire 124 is not particularly limited, it is, for example, three to twelve, preferably five to eight.

Examples of the materials for insertion tube 130, movable wire 124, and fixing wires 126 are stainless steel such as SUS304, a resin such as polyamide and PTFE, and stainless steel coated with a resin, respectively. In particular, movable wire 124 preferably has a line strength high enough to withstand load at the time of exclusion, for example, 1750 MPa or more, preferably 2100 MPa or more.

Cap 132, movable wire 124, and fixing wires 126 constituting excluding portion 122 and insertion tube 130 preferably have a smooth surface to prevent damage to the organ. Further, in order to prevent sparks generated between these components and any other instrument during the surgery, a coating material having electrical insulating properties may be applied to such surfaces. Such a coating material can be a material normally used for coating of a medical instrument as in the case of the coating material for perforation tube 110, and the same applies to the thickness of the coating layer.

The length of undeveloped excluding portion 122 accommodated in perforation tube 110 varies depending on, for example, the size of a to-be-designed retractor, and accordingly, is not necessarily limited. In one embodiment, the length of the excluding portion accommodated in perforation tube 110 is, for example, 20 mm to 100 mm, preferably 40 mm to 80 mm, and is 50 mm in the present embodiment. The size of the outside diameter of excluding portion 122 accommodated in perforation tube 110 is, for example, 1 mm to 3 mm, preferably 1.4 mm to 2.2 mm, and is 1.8 mm in the present embodiment.

The length and width (e.g., diameter) of excluding portion 122 in the most developed state vary depending on, for example, the size of the entire to-be-designed retractor, and accordingly, is not necessarily limited. In one embodiment, the length of excluding portion 122 is, for example, 25 mm to 80 mm, preferably 35 mm to 55 mm. In one embodiment, the maximum width of excluding portion 122 is, for example, 10 mm to 80 mm, preferably 15 mm to 45 mm, and is 30 mm in the present embodiment. Note that the width of excluding portion 122 refers to the length by which excluding portion 122 contacts an exclusion target in, for example, the direction orthogonal to the longitudinal direction of the movable wire. In the retractor shown in FIG. 1, in the present embodiment, since the fixing wires spread to be located on substantially the same circumference in the cross-section orthogonal to the longitudinal direction of the movable wire, the diameter of the circle formed by the fixing wires is taken as the width of excluding portion 122. Also, the width (diameter) of excluding portion 122 which is developed so as to have the largest diameter is taken as a maximum width (maximum diameter).

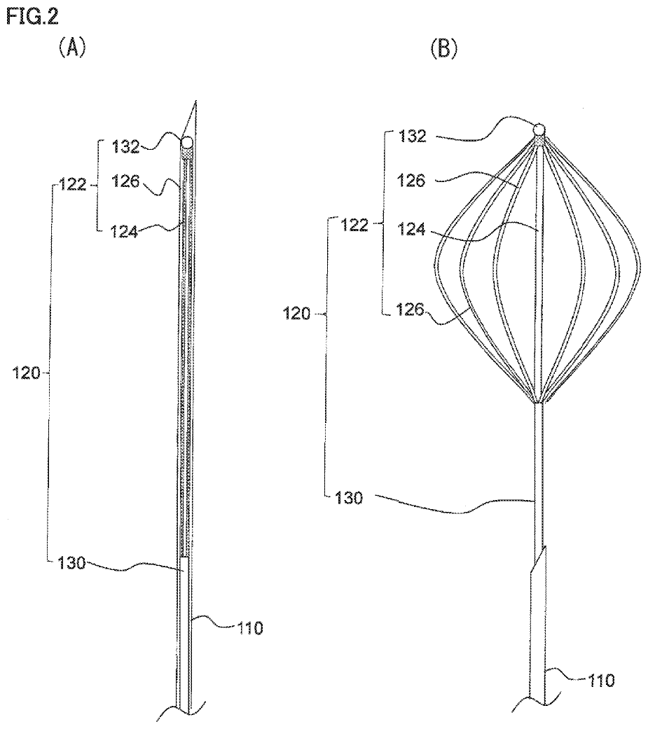

FIG. 2 schematically shows an example developing body 120 of the retractor of the present invention. As shown in FIG. 2(A), in developing body 120, the distal end of movable wire 124 and the distal ends of the respective fixing wires 126 are joined to each other by cap 132 disposed at the distal end (tip) of excluding portion 122. The proximal ends of the respective fixing wires 126 are fixed with insertion tube 130, and movable wire 124 passes through insertion tube 130. When developing body 120 is accommodated in perforation tube 110 as shown in FIG. 2(A), fixing wires 126 keep extending straight. Consequently, excluding portion 122 of developing body 120 has the most contracted state, which is a linear shape in the present embodiment, in the radial direction of perforation tube 110, and can freely slide in perforation tube 110.

As shown in FIG. 2(B), with excluding portion 122 of developing body 120 extended from the tip of perforation tube 110, when the distal end of insertion tube 130 is brought close to cap 132, fixing wires 126 each having an end fixed to cap 132 and an end fixed to the distal end of insertion tube 130 deflect in the direction in which fixing wires 126 are away from the axis of movable wire 124 (radially), so that a cocoon-shaped structure can be built by the whole of fixing wires 126 as excluding portion 122. Excluding portion 122 developed in the cocoon shape excludes a target such as the inside wall of the hollow organ or the organ in the body cavity, and can define predetermined space in the hollow cavity or the body cavity.

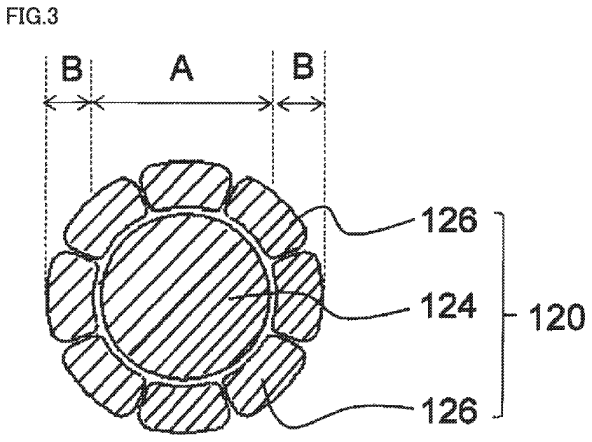

FIG. 3 schematically shows example cross-sections of a movable wire and fixing wires in the direction orthogonal to the long axis of developing body 120 of retractor 100 of the present embodiment, which shows the cross-section of excluding portion 122 which is undeveloped.

For a diameter A of movable wire 124 and a thickness B of fixing wire 126 in FIG. 3, the length ratio (A/B) preferably satisfies, for example, 1 to 10, preferably 2 to 7 to allow fixing wire 126 to be developed easily and to provide sufficient strength to each wire 124, 126. In one embodiment, A is 0.8 mm and B is 0.365 mm (A/B is 2.2) in the configuration of a relatively thin wire. In the configuration of a thicker wire, for example, A is 1.3 mm and B is 0.365 mm (A/B is 3.6).

In FIG. 3, though the cross-section of movable wire 124 of excluding portion 122 is substantially circular, it is not necessarily limited to such a shape. Shapes of a circle, a regular polygon (e.g., square, regular hexagon, regular octagon) and the like are preferred because the force applied in developing of fixing wires 126 is distributed uniformly. Fixing wires 126 having substantially the same cross-sectional shape are arranged around movable wire 124. The surfaces of fixing wires 126 which contact movable wire 124 preferably have a shape that matches the outside diameter of movable wire 124. This avoids an unnecessary space defined between fixing wires 126 and movable wire 124, and the total volume of excluding portion 122 during accommodation can be made as small as possible.

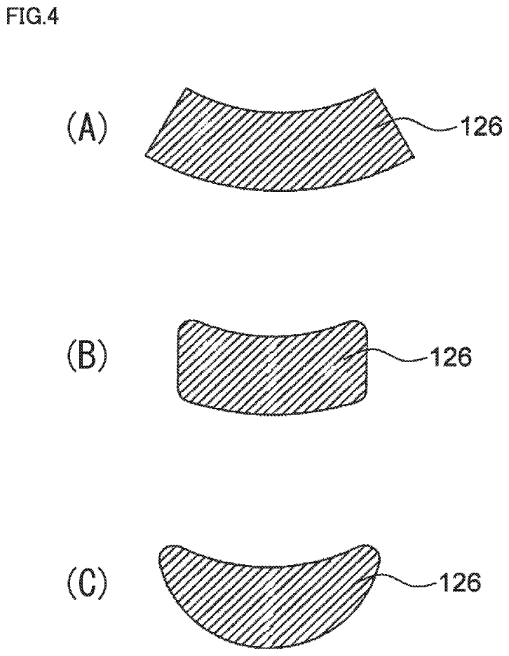

More specifically, the cross-section of fixing wire 126 has the shape of a partial circular ring in which part of the cross-section as shown in FIGS. 4(A) to 4(C) substantially coincides with part of the outer circumference of the cross-section of movable wire 124. In other words, the cross-section of fixing wire 126 may have a shape of part of an arc which is simply cut out of the arc ring, as shown in FIG. 4(A), may have a blunt shape in which four corners of the partial circular ring shown in FIG. 4(A) are rounded to reduce damage to the surrounding tissues at the time of exclusion, as shown in FIG. 4(B), or may have a crescent shape in which a portion of fixing wire 126, which corresponds to the outer edge side of the excluding portion, is rounded further to be blunt, as shown in FIG. 4(C). The cross-sections of fixing wires 126 are preferably the same when development is performed uniformly.

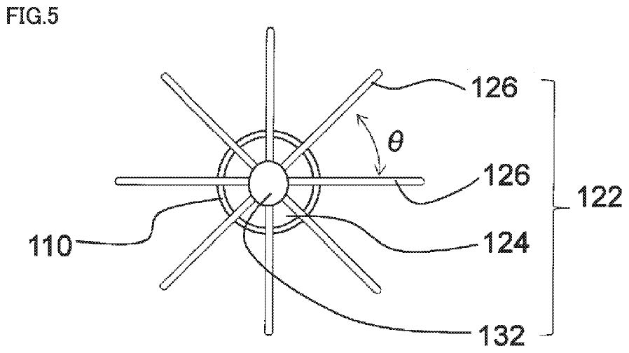

FIG. 5 is a schematic view of retractor 100 of the present invention as seen from the distal end side of the retractor when developing body 120 is extended from perforation tube 110 and is developed.

As shown in FIG. 5, when developing body 120 is developed, fixing wires 126 preferably have a substantially equal developing angle (θ) therebetween around movable wire 124. In other words, the developing angle (θ) can be set in accordance with the number of fixing wires 126 of excluding portion 122. As fixing wires 126 are developed at substantially the same developing angle (θ) as shown in FIG. 5, movable wire 124 can maintain a straight shape in the axial direction without warping.

In retractor 100 of the present embodiment, grip 160 provided on the proximal end side of retractor 100 controls the extension of developing body 120 from perforation tube 110, accommodation of developing body 120 in perforation tube 110, and change to the developed or undeveloped state of excluding portion 122 in developing body 120.

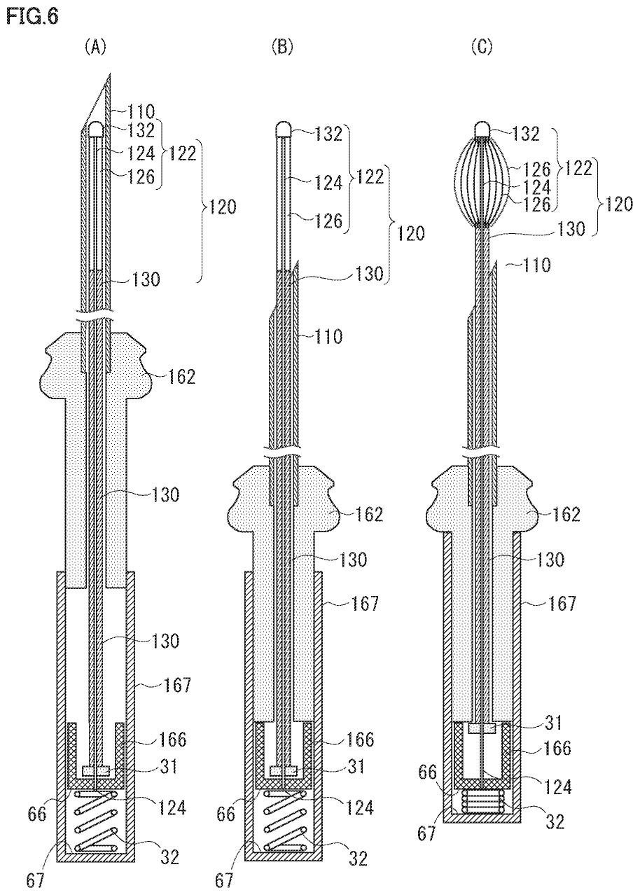

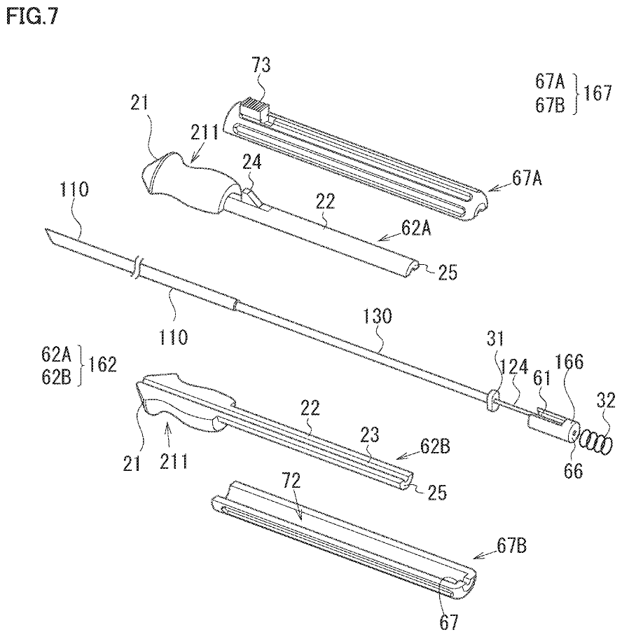

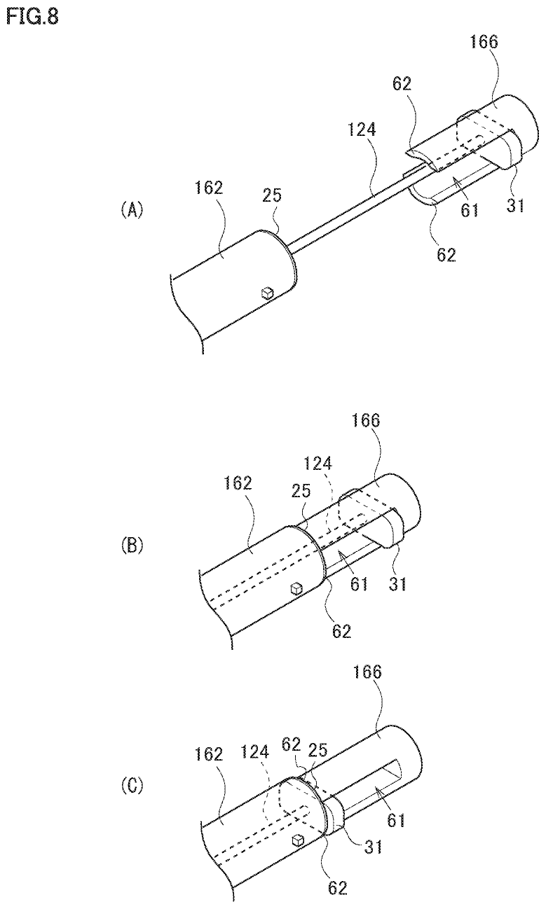

FIG. 6 schematically shows cross-sections of retractor 100. FIG. 7 is an exploded perspective view of retractor 100. FIG. 8 shows proximal ends of insertion tube 130 and movable wire 124 and therearound. As shown in FIG. 6(A), the proximal end of perforation tube 110 is connected to first grip portion 162. First grip portion 162 is composed of an upper portion 62A and a lower portion 62B, which are obtained by bisecting first grip portion 162 longitudinally, as shown in FIG. 7, and the proximal end portion of perforation tube 110 is sandwiched between upper portion 62A and lower portion 62B, and perforation tube 110 is fixed with a screw (not shown) or the like so as not to move relative to first grip portion 162.

First grip portion 162 includes a grip 21 on the distal side and includes a base 22, which has a diameter smaller than that of grip 21 and has a column shape, on the proximal side. Grip 21 has a distal end surface 210, which has a tapered shape, and a narrow portion 211, which is formed at substantially the center in the longitudinal direction and has a diameter smaller than that of any other portion, leading to an easy-to-operate configuration that allows the finger(s) of the operator to be caught by the tapered distal end surface 210 or narrow portion 211 when the operator grips grip 21. First grip portion 162 has a hollow 23 passing therethrough from the distal end to the proximal end. Hollow 23 is in communication with the interior space of perforation tube 110 attached to the distal side of the first grip, and developing body 120 is inserted into the interior space of perforation tube 110 from hollow 23.

Second grip portion 167 is a tubular member composed of an upper portion 67A and a lower portion 67B, which are obtained by bisecting second grip portion 167 longitudinally as shown in FIG. 7, and is fitted around a base 22 of first grip portion 162 and can slide (advance or retract) toward the distal end or the proximal end along base 22. In the present embodiment, since second grip portion 167 is positioned on the proximal side of first grip portion 162 as described above, for example, the operator can hold second grip portion 167 with the little finger (fifth finger) or the ring finger (fourth finger) and hold first grip portion 162 with the index finger (second finger) or the thumb (first finger) to operate these grip portions with one hand. Although the present embodiment has described the example in which second grip portion 167 is fitted around first grip portion 162, the present invention is not limited thereto. First grip portion 162 may be slidably fitted around second grip portion 167. An engaging pawl 24 is provided on the distal side of base 22 of first grip portion 162, that is, near grip 21. When second grip portion 167 is moved to the distal side until it collides with grip 21, engaging pawl 24 and an engaging recess (not shown) provided inside second grip portion 167 engage with each other, thereby restraining the movement of second grip portion 167. In other words, engaging pawl 24 forms a lock mechanism that restricts the movement of second grip portion 167. A release portion 73 is provided outside of the fitting recess in second grip portion 167. As release portion 73 is pushed inwardly with engaging pawl 24 and the fitting recess engaging with each other, engaging pawl 24 comes out of the fitting recess to release the engagement, allowing second grip portion 167 to move.

A flange 31, which projects in the direction orthogonal to the longitudinal direction of insertion tube 130, is connected to the proximal end of insertion tube 130, so that flange 31 is fixed to an inside wall 72 of second grip portion 167. In other words, insertion tube 130 has a proximal end connected to second grip portion 167, and when second grip portion 167 is moved to advance or retract along base 22 of first grip portion 162, insertion tube 130 advances or retracts together with second grip portion 167.

A restriction member 166, which abuts against proximal end surface 25 of first grip portion 162 to restrict the movement of movable wire 124, is provided at the proximal end of movable wire 124 which is provided to pass through insertion tube 130. Restriction member 166 is a column-shaped member that is accommodated in the interior space of second grip portion 167 and can advance or retract in second grip portion 167, and includes, on the distal side, a groove 61 in which flange 31 can be accommodated. Since restriction member 166 is connected with movable wire 124, movable wire 124 is connected with fixing wires 126, and fixing wires 126 are connected with second grip portion 167 via insertion tube 130, restriction member 166 is moved to the distal side or the proximal side along with the movement of second grip portion 167. FIG. 8(A) shows the state in which a distal end 62 of restriction member 166 is not held in contact with first grip portion 162 and restriction member 166 does not restrict the movement of movable wire 124. In this state, flange 31, that is, the proximal end of insertion tube 130 is accommodated in groove (hollow) 61 of restriction member 166, and distal end 62 of restriction member 166 is positioned on the distal side relative to flange 31 and the proximal end of insertion tube 130. For this reason, when second grip portion 167 is moved to the distal side from the state of FIG. 8(A), distal end 62 of restriction member 166 abuts against first grip portion 162 before second grip portion 167 is located most to the distal side, thus restricting the movement of movable wire 124. FIG. 8(B) shows the state in which distal end 62 of restriction member 166 is held in contact with first grip portion 162, and restriction member 166 restricts the movement of movable wire 124. FIG. 8(B) also shows the state in which flange 31 and the proximal end of insertion tube 130 are positioned at the proximal portion of groove 61 as in FIG. 8(A). In this state, excluding portion 122 is undeveloped as described below. Further from the state of FIG. 8(B), second grip portion 167 can be moved to the distal side until flange 31 abuts against proximal end surface 25 of first grip portion 162 as shown in FIG. 8(C). Although groove 61 in which flange 31 is accommodated is provided in restriction member 166 in the present embodiment, the present invention is not limited thereto. A groove in which flange 31 is accommodated may be provided on the proximal end side of first grip portion 162. In this case, the restriction member may be, for example, a disk-shaped member in which the distal end has a circular appearance, and as flange 31 is accommodated in the groove of first grip portion 162, the restriction member abuts against the proximal end of first grip portion 162 to restrict the movement of the movable wire. Inside of second grip portion 167 and on the proximal side of restriction member 166, an elastic member 32 is provided. In other words, elastic member 32 is provided between inside wall 67 of the closed proximal end of tubular second grip portion 167 and proximal end surface 66 of restriction member 166. In the state in which second grip portion 167 is moved most to the distal side, thus, the elastic member is pushed to be shrunk between inside wall 67 of second grip portion 167 and proximal end surface 66 of restriction member 166. Elastic member 32 of the present embodiment is a coil spring that elastically deforms in the longitudinal direction of movable wire 124, that is, the direction in which the second grip portion advances or retracts.

The action in the use of retractor 100 of the present invention which has the above configuration will now be described. Retractor 100 is inserted into the inside wall of the hollow organ or the organ in the body cavity as the distal end of the perforation tube 110 perforates through a trocar provided in an incision or through direct perforation.

Then, second grip portion 167 is pushed toward first grip portion 162 to the distal end side, that is, the first grip portion 162 side. This causes insertion tube 130 connected to second grip portion 167 to be pushed to the distal side, so that fixing wire 126 connected to the distal end of insertion tube 130 and movable wire 124 connected to the distal end of fixing wire 126 move together to the distal side. Thus, excluding portion 122 of developing body 120 is extended from the tip of perforation tube 110, as shown in FIGS. 1(B) and 6(B).

When movable wire 124 is pushed to a predetermined position as shown in FIG. 6(B), distal end 62 of restriction member 166 abuts against proximal end surface 25 of first grip portion 162, thus restricting the movement of movable wire 124 further to the distal side.

By further pushing second grip portion 167 to the distal side from this state, only insertion tube 130 slides to the distal side relative to perforation tube 110 and movable wire 124. Thus, with the positional relationship between movable wire 124 and fixing wires 126 maintained in the cap 132 portion, only the proximal ends of fixing wires 126 joined to the distal end of insertion tube 130 slide to the distal side, so that fixing wires 126 are curved outwardly. As a result, fixing wires 126 are developed in excluding portion 122, so that a cocoon shape is formed. The thus developed excluding portion 122 can exclude a target.

The size of excluding portion 122 to be developed can be changed freely by adjusting the position of second grip portion 167 between the state in which distal end 62 of restriction member 166 is held in contact with proximal end surface 25 of first grip portion 162, which is shown in FIG. 6(B), and the state in which second grip portion 167 is moved most to the distal side, which is shown in FIG. 6(C).

When second grip portion 167 is moved to a predetermined position, is moved to the distal side to a position at which second grip portion 167 abuts against grip 21 in the present embodiment, engaging pawl 24 engages with the engagement recess of second grip portion 167. This stops advancement or retraction of second grip portion 167, so that excluding portion 122 is kept being developed. Thus, excluding portion 122 can be kept being developed even when the operator does not maintain the position of second grip portion 167, facilitating exclusion. Although the present embodiment has described the example in which engaging pawl 24 is provided at one location, engaging pawls 24 may be provided at a plurality of locations such that the developed excluding portion 122 can be maintained in different sizes.

When second grip portion 167 is moved further to the distal side from the state shown in FIG. 6(B), elastic member 32 is pushed to be shrunk between inside wall 67 of second grip portion 167 and proximal end surface 66 of restriction member 166 (e.g., FIG. 6(C)), so that second grip portion 167 is biased toward the distal side by the elastic force of elastic member 32. Thus, when the user stops moving second grip portion 167 by, for example, removing the hand therefrom, second grip portion 167 is moved to the distal end by the elastic force of elastic member 32, returning to the state of FIG. 6(B). In other words, excluding portion 122 is closed (shut). Thus, as excluding portion 122 is developed, excluding portion 122 can be closed by the elastic force of elastic member 32 even when the user does not actively perform an operation of returning second grip portion 167, facilitating the operation. In particular, when excluding portion 122 is kept being developed by engaging pawl 24, as release portion 73 is pushed to be disengaged from engaging pawl 24, excluding portion 122 is closed by the elastic force of elastic member 32. That is to say, the user can close excluding portion 122 by a single-touch operation of merely pushing release portion 73. Although the present embodiment has described the example in which elastic member 32 is provided between inside wall 67 of tubular second grip portion 167 and proximal end surface 66 of restriction member 166, the arrangement of elastic member 32 is not limited thereto. For example, it suffices that elastic member 32 is arranged between, for example, the proximal end surface of first grip portion 162 and the distal end surface of flange 31 to keep second grip portion 167 away from first grip portion 162, from the state in which second grip portion 167 is brought close to first grip portion 162 as shown in FIG. 6(C) to the state shown in FIG. 6(B), that is, to the position at which excluding portion 122 is closed.

A conventional retractor moves the movable wire and the insertion tube to the distal side to extend the developing body from the perforation tube while being gripped so as not to move a perforation tube, and moves the insertion tube to the distal side to develop the excluding portion of the developing body while being gripped so as not to move the perforation tube and the movable wire. Thus, the conventional retractor requires the operations of three components, namely, a perforation tube, a movable wire, and an insertion tube, and accordingly, cannot be operated with one hand. Also, since the three components move independently, the user needs to perform an operation while checking the positional relationship of the three components. For example, the excluding portion cannot be developed appropriately if the user tries to develop the excluding portion while the developing body is not extended from the perforation tube sufficiently. Thus, the user has performed surgery while checking the positional relationship of these components with the hands as necessary.

Contrastingly, in retractor 100 of the present embodiment, the operation of restricting the movement of movable wire 124 in development of excluding portion 122 is performed by restriction member 166. This requires the user to merely operate two grip portions, first grip portion 162 and second grip portion 167, enabling an operation with one hand.

In particular, retractor 100 of the present embodiment can perform the operation of extending the developing body and the operation of developing the developing body by merely adjusting the distance between first grip portion 162 and second grip portion 167, facilitating exclusion. Further, since the user can determine the distance between first grip portion 162 and second grip portion 167 through a touch when the user grips first grip portion 162 and second grip portion 167 with one hand, the user has no need to check the positional relationship thereof with the hands, and therefore, can concentrate on the surgery without having to remove the eyes off the monitor of an endoscope.

The retractor of the present embodiment is used to perform exclusion in various types of surgery of the hollow organs such as stomach, small intestine, large intestine, and vagina, as well as other organs such as liver, pancreas, kidney, gallbladder, spleen, uterus, and lungs.

For example, in the surgery of the stomach or esophagus, the retractor of the present invention is inserted to the lower surface of the hepatic left lobe interfering with the operative field to elevate the reactor toward the abdomen. This eliminates the interference with the operative field, allowing the surgery to be performed safely and efficiently. Similarly, in the operation of the pelvic floor, the surgical operation can be efficiently performed around the rectum by spreading the uterus with the retractor of the present embodiment.

The present embodiment can provide a retractor capable of freely excluding a target. In particular, the present embodiment can provide an easy-to-handle retractor that can develop excluding portion 122 for exclusion through operations of two grip portions 162 and 167, not three grip portions as described in WO 2015/079719. Further, the present invention can ensure the field of view and the operation space without gas injection into the body cavity, which is beneficial in that it allows for, for example, gasless surgery.

### Second Embodiment

Although the first embodiment has described the configuration in which fixing wires are arranged equidistantly on the circumference having its center at the movable wire in the cross-section orthogonal to the longitudinal direction of the movable wire and the fixing wires are developed to form a complete cocoon shape, the present embodiment differs from the first embodiment in the configuration in which some of the fixing wires are omitted. Since the other components are the same, the same components are for example denoted by the same references, description of which is not repeated again.

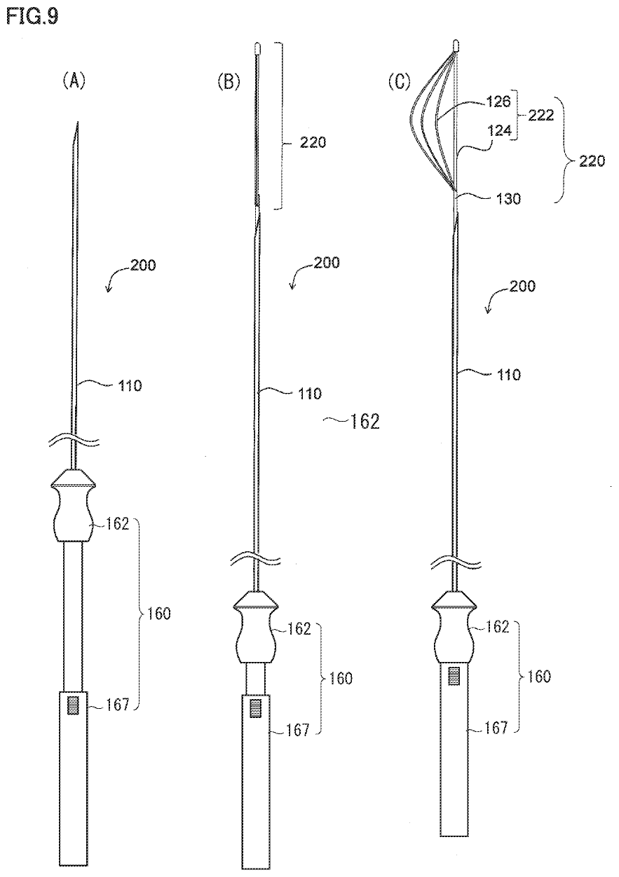

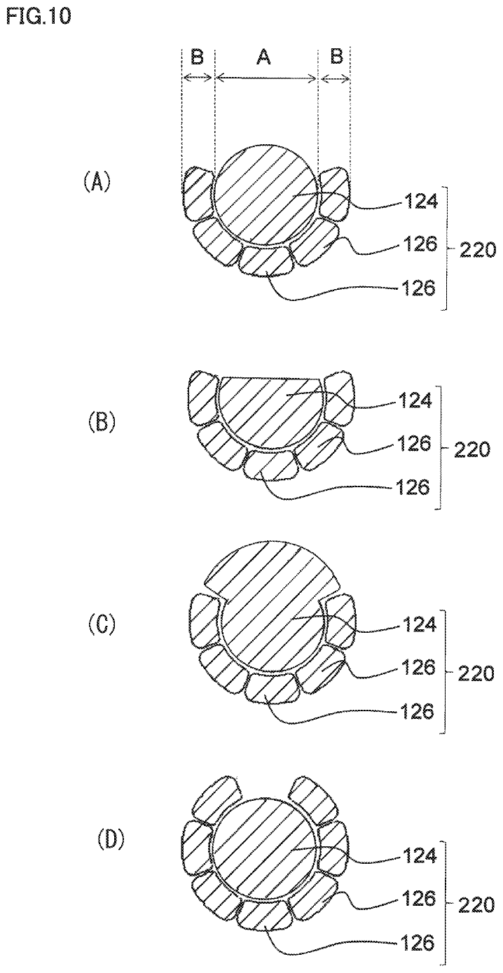

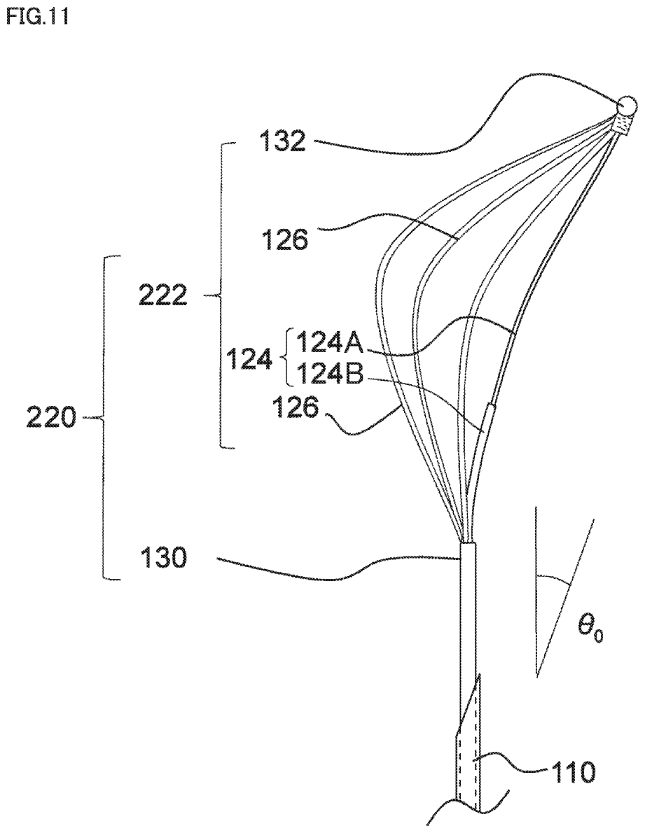

FIG. 9 shows an example retractor 200 according to the present embodiment, where FIG. 9(A) shows the state in which a developing body 220 is accommodated in perforation tube 110, FIG. 9(B) shows the state in which developing body 220 is extended from the tip of perforation tube 110, and FIG. 9(C) shows the state in which excluding portion 222 of developing body 220 is developed. FIG. 10 shows example cross-sections of excluding portion 222 (the cross-sections of movable wire 124 and fixing wires 126) in the direction orthogonal to the long axis of developing body 220 of retractor 200 of the present embodiment. FIG. 11 shows the state in which excluding portion 222 is opened in detail.

As shown in FIGS. 9 to 11, in retractor 200 of the present embodiment, fixing wires 126 are arranged around only part of movable wire 124, and no wires are arranged around the other part of movable wire 124.

Movable wire 124 may have a substantially circular cross-section as shown in FIG. 10(A), may have a cross-section having a shape (e.g., the shape of the quarter of the moon) having a combination of a circular arc and a chord as shown in FIG. 10(B), or may have a cross-section of a shape having a combination of a circular arc and a chord with a protrusion on part of the chord as shown in FIG. 10(C). The shapes of equilateral polygons (e.g., square, regular hexagon, regular octagon) may be provided as other cross-sectional shapes. Fixing wires 126 having substantially the same cross-sectional shape are arranged around movable wire 124, except for around part of movable wire 124. In FIGS. 10(A) to 10(C), five fixing wires 126 are arranged on the circumference having its center at movable wire 124. The length (width) of each fixing wire 126 in the circumferential direction is one-eighth of the circumference, where fixing wires 126 are arranged in the portion corresponding to five-eighths of the circumference and no fixing wires 126 are arranged in the portion corresponding to three-eighths of the circumference.

Since developing body 220 of the present embodiment has fixing wires 126 arranged asymmetrically in the cross-section of FIG. 4 as described above, when developing body 220 is opened, a bending force acts on the portion around movable wire 124, where no fixing wires 126 are arranged, so that movable wire 124 can build a deflected shape by itself in the axial direction of perforation tube 110, more specifically, over the entire portion of movable wire 124 which corresponds to excluding portion 222 (FIG. 11). Further, such deflection causes excluding portion 222 to bend to the axial direction of perforation tube 110 on the proximal side of excluding portion 222 (i.e., near the distal end of insertion tube 130). The deflection of movable wire 124 and the bending of excluding portion 222 lead to an increased size (substantial volume) of the retraction, further increasing the operability of the retractor. The retractor in which excluding portion 222 can bend to the axial direction of perforation tube 110 can be manufactured through selection of the diameter (in particular, the diameters of a distal portion 124A and a proximal portion 124B), material, cross-sectional shape of movable wire 124 and the arrangement of fixing wires 126 as shown in FIGS. 10(A) and 10(B).

In particular, the diameter of movable wire 124 of the present embodiment in the cross-section orthogonal to the longitudinal direction differs between distal portion 124A and proximal portion 124B other than distal portion 124A, and as shown in FIG. 11, distal portion 124A is formed to be thinner than proximal portion 124B. In the present embodiment, the diameter of distal portion 124A is 0.8 mm, and the diameter of proximal portion 124B is 1.3 mm. Distal portion 124A of movable wire 124 is formed to be thinner as described above to increase the angle of bending of excluding portion 222 and to provide flexibility to the distal portion of excluding portion 222, and strength is guaranteed by proximal portion 124B. This leads to a structure that withstands the load in exclusion. Movable wire 124 having different diameters as described above can be produced by, for example, connecting the ends of wires having different diameters by, for example, welding or brazing. Besides, movable wire 124 may be produced by reducing the diameter of distal portion 124A of movable wire 124 by plastic working such as swaging.

In the present embodiment, a bending angle θ0 (FIG. 11) of excluding portion 222 is, for example, 5° to 45°, preferably 10° to 30°. Such bending angle θ0 of excluding portion 222 can be adjusted through, for example, selection of a movable wire having a strength lower than that of fixing wire 126 or an elastic movable wire as movable wire 124.

More specific examples are as follows:

(1) a stranded wire is used for movable wire 124;

(2) the cross-section of movable wire 124 is processed to, for example, an odd shape as shown in FIG. 10(B) (e.g., is processed to be flat in the direction in which bending of excluding portion 222 is desired);

(3) the sectional area of movable wire 124 is set to be smaller than the sectional area of fixing wire 126 (a thinner wire is used for movable wire 124);

(4) a material having a low Young's module, such as Ni—Ti alloy, is used for movable wire 124;

(5) a wire which is deflected in advance in excluding portion 222 is used for movable wire 124;

(6) distal portion 124A is formed to be thinner than proximal portion 124B such that the ratio of the diameter of distal portion 124A to the diameter of proximal portion 124B is not greater than a predetermined value; and

(7) a combination of some of (1) to (7) above.

Alternatively, in order to avoid such bending of excluding portion 222, for example, deflection of movable wire 124 toward the periphery of movable wire 124 around which no fixing wires 126 are arranged can be prevented or reduced by processing a wire shaped to have a projection in part of the cross-section as shown in FIG. 10(C).

Further, in the present invention, the number and arrangement of fixing wires 126 are selected with respect to movable wire 124 such that among the developing angles formed between two adjacent fixing wires, one developing angle θ2 is greater than the other developing angles θ1 and developing angle θ2 has a predetermined angle in the excluding portion at the time of development of the developing body.

In the present embodiment, the term “developing angle” refers to an angle formed between one fixing wire 126 and its adjacent fixing wire 126 as excluding portion 222 in development of body 220 of retractor 200 is viewed from the distal end side of retractor 200.

In retractor 200 of the present embodiment, such developing angle θ2 is 90° to 240°, preferably 120° to 240°, more preferably 120° to 180°. The other developing angles (θ1) may be set to substantially the same angle or to different angles as long as all of the other developing angles θ1 are smaller than developing angle θ2.

The relationship between fixing wires 126 and developing angles θ1 and 02 will be described using a more specific example.

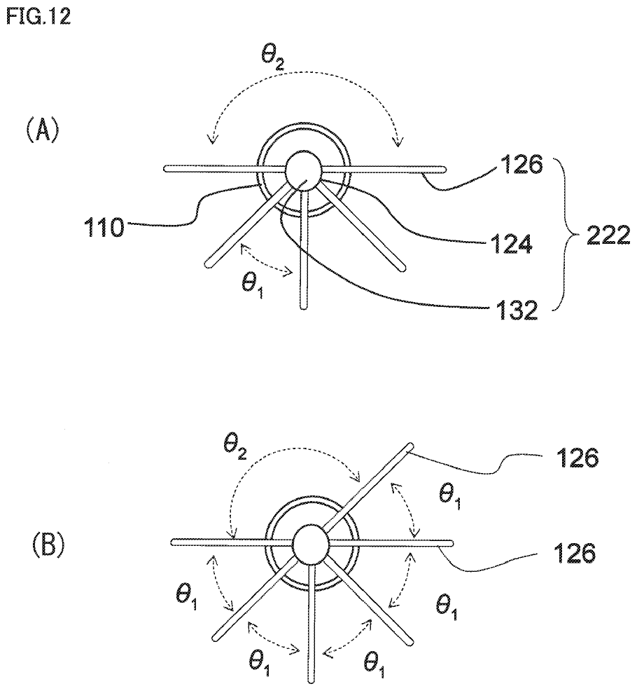

FIG. 12 schematically shows an example distal end tip of retractor 200 of the present embodiment when developing body 220 is extended from perforation tube 110 and is deployed in retractor 200.

In the example shown in FIG. 12(A), fixing wires 126 of excluding portion 222 of retractor 200 are five fixing wires. Among the developing angles formed between two adjacent fixing wires 126, one developing angle θ2 is approximately 180°, and the other developing angles θ1 are approximately 45°. In the example shown in FIG. 12(B), fixing wires 126 of excluding portion 222 of retractor 200 are six fixing wires 126. Among the developing angles formed between two adjacent fixing wires 126, one developing angle θ2 is approximately 135°, and the other developing angles θ1 are approximately 45°.

In the present embodiment, developing angle θ2 and the other developing angles θ1 satisfy the above relationship, thus forming a retraction area with improved workability between two fixing wires having developing angle θ2 in excluding portion 222.

1. A retractor comprising:
an outer tube having a tube shape;
a developing body inserted into the outer tube;
a first grip portion connected to a proximal end of the outer tube; and
a second grip portion that is connected to the developing body and moves relative to the first grip portion to move the developing body and the first grip portion relative to each other, wherein
the developing body includes
an excluding portion including a movable wire and a plurality of fixing wires arranged around the movable wire, a distal end of the movable wire and distal ends of the fixing wires being connected to each other,
an insertion tube having a distal end connected to proximal ends of the fixing wires and a proximal end connected to the second grip portion, the insertion tube allowing the movable wire to pass therethrough, and
a restriction member that is connected to a proximal end of the movable wire and restricts a movement of the movable wire,
the restriction member included in the developing body restricts the movement of the movable wire when the developing body and the first grip portion move relative to each other through the relative movement of the first grip portion and the second grip portion to cause the restriction member to abut against the first grip portion, and
when the first grip portion and the second grip portion are moved relative to each other so as to be close to each other with the movement of the movable wire restricted, the excluding portion is developed by bringing the distal end of the insertion tube connected with the second grip portion and the distal end of the movable wire, the movement of which is restricted, close to each other to deflect the fixing wire.

2. The retractor according to claim 1, wherein when the first grip portion and the second grip portion are moved relative to each other so as to be close to each other with the developing body accommodated in the outer tube, the developing body connected with the second grip portion and the outer tube connected with the first grip portion are moved relative to each other to cause the developing body to extend from a tip of the outer tube.

3. The retractor according to claim 1, wherein the first grip portion and the second grip portion are slidably fitted with each other, and the first grip portion and the second grip portion are moved relative to each other through sliding of the first grip portion and the second grip portion.

4. The retractor according to claim 1, comprising a lock mechanism that locks the second grip portion at a position at which the second grip portion is moved to a distal side and the excluding portion is developed.

5. The retractor according to claim 1, wherein the outer tube is a perforation tube having a distal end projecting to a distal side at an acute angle.
